(19) United States
(12) Patent Application Publication
(10) Pub. No.: US 2007/0087056 A1
(43) Pub. Date: Apr. 19, 2007

(54) Pharmaceutical form with sustained pH-independent active ingredient release for active ingredients having strong pH-dependent solubility

(76) Inventors: Claudia Guthmann, Berlin (DE); Heiko Kranz, Berlin (DE); Ralph Lipp, Zionsville, IN (US); Torsten Wagner, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/500,501

(22) Filed: Aug. 8, 2006

Related U.S. Application Data
(60) Provisional application No. 60/706,428, filed on Aug. 9, 2005.

(51) Int. Cl. A61K 9/24

(52) U.S. Cl. 424/472

(57) ABSTRACT

Solid pharmaceutical formulation for a sustained pH-independent active ingredient release comprising at least one layer of one or more water-insoluble polymers, at least one layer of one or more pH-dependently water-soluble polymers and an active ingredient-containing core, having strong pH-dependent water solubility and comprises at least one osmagent.

This application claims the benefit of the filing date of U.S. Provisional Application Ser. No. 60/706,428 filed Aug. 9, 2006.

### TECHNICAL FIELD

The invention relates to a solid pharmaceutical formulation for a sustained pH-independent active ingredient release comprising at least one layer of one or more water-insoluble polymers, at least one layer of one or more pH-dependent water-soluble polymers and an active ingredient-containing core, where the core comprises an active ingredient having strong pH-dependent water solubility and at least one osmotically active ingredient.

Active ingredients having strong pH-dependent water solubility are for example substances which have very poor solubility at basic pH values, normally having a solubility in water of less than 0.1 mg/ml, whereas the solubility at acidic pH values (pH<4) extends up to values of 1 mg/ml or higher.

Generally pH-dependent water-soluble active ingredients can also be defined as substances having a difference of at least 10-fold in the water solubility at acidic and basic pH values.

One example of an active ingredient having strong pH-dependent solubility in water is (2R)-1-((4-chloro-2-(ureido)phenoxy)methyl)carbonyl-2-methyl-4-(4-fluoro-benzyl)piperazine or a salt thereof.

(2R)-1-((4-chloro-2-(ureido)phenoxy)methyl)carbonyl-2-methyl-4-(4-fluorobenzyl)piperazine is called piperazineurea hereinafter and has the following structure: 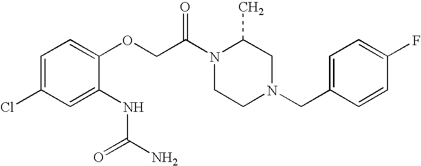
(2R)-1-((4-chloro-2-(ureido)phenoxy)methyl)carbonyl-2-methyl-4-(4-fluorobenzyl)piperazine and its salts are prepared by the method described in Example 2 in WO 98/56771.

Salts thereof are, for example, the hydrochloride, dihydrogen phosphate, hydrogen sulphate, sulphate, mesylate, ethylsulphonate, malate, fumarate and tartrate.

The following invention further relates to a matrix pellet for a sustained pH-independent active ingredient release comprising at least one layer of one or more water-insoluble polymers in which the pore-forming substances are present and are dissolved out after contact with the aqueous medium and thus form a microporous membrane, and comprising at least one layer of one or more pH-dependently water-soluble polymers, and an active ingredient-containing core, where the core comprises piperazineurea and at least one water-soluble ionic substance from the group of magnesium chloride, magnesium sulphate, lithium chloride, sodium chloride, potassium chloride, lithium sulphate, sodium sulphate, potassium sulphate, lithium phosphate, sodium phosphate, potassium phosphate, ammonium chloride, ammonium sulphate, ammonium phosphate as osmagent.

Further solid pharmaceutical formulations within the meaning of the invention are single-unit systems such as, for example, tablets and multiparticulate systems. Multiparticulate systems may be for example granular particles, pellets or mini tablets. These may be packed into hard or soft gelatin capsules, and compressed to tablets. The original formulation usually disintegrates into many subunits in the stomach. The minidepots then gradually pass from the stomach into the intestine. The minidepots are moreover normally able to pass through the pylorus when the sphincter is closed.

Sustained release formulations are medicaments which can be administered orally and have a longer-lasting effect of the medicament. In these cases, the active pharmaceutical ingredient is released slowly.

### PRIOR ART

Various pharmaceutical formulations for controlled active ingredient release are present in the literature.

An elementary osmotic pump (EOP), for example, are tablets which consist of an osmotically active tablet core which is coated with a semipermeable membrane which comprises a release orifice.

The tablet core may comprise an osmotically active medicinal substance or, in the case of a medicinal substance of low osmotic activity, osmotically active additives, also generally defined as osmagents. Water flowing through the semipermeable membrane (SPM) into the pharmaceutical form generates a hydrostatic pressure which forces the dissolved medicinal substance through the release aperture.

The object of an EOP is controlled active ingredient release, achieving 0 order release kinetics. Thus, the amount of medicinal substance released from the pharmaceutical form per unit time remains the same.

A precondition for an EOP is a moderately water-soluble active ingredient.

Push and pull osmotic pumps (PPOPs) have been established also to allow controlled release of slightly soluble medicinal substances.

These comprise multichamber tablet systems whose core comprises an osmotic active ingredient compartment and a swellable osmotically active polymer, with the two compartments being separated by an elastic diaphragm. The entire tablet core is in turn enveloped by an SPM which comprises a release orifice on the active ingredient containing side.

Water penetrates into both compartments, whereupon the polymer swells and thus forces the diaphragm into the active ingredient compartment. The active ingredient is then delivered through the release aperture. The aim in this case too is to create plasma levels which remain the same owing to the 0 order active ingredient release.

Hence, systems which operate osmotically, such as the elementary osmotic pump (EOP) and push and pull osmotic pumps (PPOP) release at least moderately water-soluble active ingredients from tablets which consist of a semipermeable membrane around an osmotically active core which comprises at least one substance having an osmotic effect (osmagent) and, in the case of the PPOP, an expanding polymer push compartment.

Since semipermeable membranes are permeable only by the medium but not by the active ingredient, the active constituent is released through at least one orifice in the semipermeable membrane.

The essential aim of osmotic pumps as known in the state of the art is 0 order active ingredient release.

In contrast to EOP and PPOP, pharmaceutical forms without semipermeable membranes have also been described, for example: Controlled Porosity Osmotic Pumps (CPOP).

CPOPs were also developed in order to replace the elaborate manufacture of the above-described systems in which release orifices must be bored by drilling machines or lasers.

These CPOP formulations have a water-insoluble polymer membrane into which water-soluble ingredients are incorporated and, after contact with the aqueous medium, are dissolved out and thus form a microporous membrane which is now permeable by medium and active ingredient.

In these systems, in detail the osmotic tablet core is enveloped by an insoluble polymer membrane into which water-soluble substances have been incorporated. After the pharmaceutical form is introduced into the medium, these water-soluble substances are dissolved out.

This results in pores through which the active ingredient release takes place. These systems also comprise tablets which show controlled release.

In these cases, the active ingredient release depends in particular on the water-solubility of the medicinal substance and thus shows a pH-dependent release for pH-dependently soluble active ingredients.

Delayed release pellet formulations have been described for osmagent-containing matrix pellet cores which have been coated with a semipermeable membrane. This membrane is stretched owing to the swelling of the core, resulting after a lag time in pores which make the membrane permeable by medium and active ingredient and thus bring about a delayed active ingredient release. Such delayed release formulations are utilized for accurately targeted active ingredient release in the GI tract or release according to chrono-pharmacological aspects or are used when the kinetics of absorption of a medicinal substance are non-linear.

Asymmetric membranes which can be applied to tablets and also to pellet cores bring about an improved release of active ingredients of low solubility. However, these formulations also do not show pH-independent active ingredient release for pH-dependently soluble substances. A pH-independent release has been described for such systems when pH adjusters have been incorporated in the core formulation for buffering.

Such excipients either acids or bases alter the pH within the formulation to such an extent that the active ingredient solubility is improved, even in pH-unfavourable media.

Further systems described in the literature for pH-independent active ingredient release by means of pH adjusters in the core of tablets or pellets are also described for systems which do not operate osmotically.

Multilayer coating combinations have been described for the combination of water-soluble and water-insoluble polymer layers, where the water-soluble polymers do not show pH-dependent solubility and thus any control of the release of pH-dependently soluble active constituents either.

There have furthermore been descriptions of combinations of water-insoluble and pH-dependently soluble polymer layers and polymer mixtures, a pH-independent active ingredient release being achieved solely on the basis of differences in the permeability of the polymer coating. The permeabilities of the polymer film can be adjusted accurately in these cases. The pH-dependently soluble polymer component always shows a contrary solubility to the active ingredient. Weak active ingredient bases are coated with an acid-insoluble polymer, whereas an alkali-insoluble polymer is used as pH-dependently soluble component in the case of weak active ingredient acids. The result is a thinner or more porous coating in the medium in which the active substance is less soluble. The diffusion barrier in the medium having lower active ingredient solubility is thus reduced, resulting in an improved active ingredient liberation.

The present invention relates to a solid pharmaceutical formulation for a sustained pH-independent active ingredient release comprising at least one layer of one or more water-insoluble polymers, at least one layer of one or more pH-dependent water-soluble polymers and an active ingredient-containing core, where the core comprises an active ingredient having strong pH-dependent solubility in water and at least one osmagent.

In a preferred form of the present invention, the layer of one or more water-insoluble polymers comprises pore-forming substances which are dissolved out after contact with the aqueous medium and thus form a microporous membrane.

In a further embodiment of the invention, the layer of one or more pH-dependent water-soluble polymers is the outer layer on the solid pharmaceutical formulation, and the layer of one or more water-insoluble polymers is the inner one.

The present invention further relates to a matrix pellet for a sustained pH-independent active ingredient release comprising at least one inner layer of one or more water-insoluble polymers in which pore-forming substances are present and, after contact with the aqueous medium, are dissolved out and thus form a microporous membrane, and comprising at least one outer layer of one or more pH-dependent water-soluble polymers, and an active ingredient-containing core, where the core comprises piperazineurea and at least one water-soluble ionic substance from the group of magnesium chloride, magnesium sulphate, lithium chloride, sodium chloride, potassium chloride, lithium sulphate, sodium sulphate, potassium sulphate, lithium phosphate, sodium phosphate, potassium phosphate, ammonium chloride, ammonium sulphate, ammonium phosphate.

Surprisingly, simply mixing water-insoluble with pH-dependently soluble polymers and application of layers thereof is insufficient for sustained pH-independent active ingredient release.

This phenomenon was observable even on application of very small amounts of pH-dependently water-soluble polymer, for example 2.5 and 5% (w/w) based on the total mass of the formulation.

Only by use of osmotically active substances according to the present invention was a pH-independent active ingredient release achieved. Only an osmotically active addition to the core formulation with a high active ingredient loading, for example up to 90% w/w, preferably up to 60% w/w, based on the mass of the core formulation brings about rapid penetration of medium into the core, followed by the formation of a saturated active ingredient solution which, driven by the osmotic pressure, is forced out of the solid pharmaceutical formulation.

It is possible in this way to increase significantly the release of the active substance in the medium with low active ingredient solubility.

Only there is the pH-dependently soluble polymer layer stretched, owing to the increased penetration in of medium, to such an extent that a significant emergence of active ingredient is in fact achieved.

According to the present invention, a pH-dependently water-soluble polymer layer is necessary even for a core with osmagent.

Additionally, according to a further preferred embodiment of the invention, pore-forming substances may be an addition to the water-insoluble membrane.

Owing to incorporated pore formers, the membrane rapidly becomes permeable not only by medium but also by the active ingredient. The rapid permeability of the water-insoluble membrane is very important in particular for active ingredients having a very low solubility in water.

It is now possible to adjust a pH-independent release of the active substance in the solid pharmaceutical formulation according to the present invention through the combination of water-insoluble polymer with or without further water-soluble substances for pore formation and a pH-dependently water-soluble polymer.

In addition, the release of the active substance from the solid pharmaceutical formulation according to the present invention is not only pH-independent but also substantially increased by comparison with known pharmaceutical formulations without osmagent in the core.

### BRIEF DESCRIPTION OF THE DRAWINGS

The invention is explained in more detail below by means of the drawing. This shows in:

FIG. 1 a preferred embodiment of the solid pharmaceutical formulation according to the present invention.

FIG. 2 a solid pharmaceutical formulation of FIG. 1 with pH-dependently water-soluble polymer layer without osmagent in the core. Formulations with pH-dependently water-soluble polymer layer without osmagent in the core show a very greatly reduced active ingredient release in the medium with the actually highest active ingredient solubility.

FIGS. 3a-3c show release investigations on a solid pharmaceutical formulation of FIG. 1 with pH-dependently water-soluble polymer layer without osmagent in the core. The release investigations were carried out in a USPXXV basket apparatus at 100 revolutions per minute and with a medium temperature of 37° C. (±0.5° C.). The media used were 0.1 N HCl and phosphate buffer of pH 6.8. Quantification took place by HPLC.

FIG. 4 a solid pharmaceutical formulation of FIG. 1 without pH-dependently water-soluble polymer layer with osmagent in the core.

FIG. 5 a solid pharmaceutical formulation of FIG. 1 with pH-dependently water-soluble polymer layer and osmagent in the core. A pH-independent active ingredient release was achieved through the introduction of osmotically active substances.

FIGS. 6a-6c and 7a-7c show release investigations on the solid pharmaceutical formulation of FIG. 1 with pH-dependently water-soluble polymer layer with osmagent in the core (FIGS. 6a-6c for Examples 2 and FIGS. 7a-7c for Examples 3). The release investigations were carried out in a USPXXV basket apparatus at 100 revolutions per minute and with a medium temperature of 37° C. (+0.5° C.). The media used were 0.1 N HCl and phosphate buffer of pH 6.8. Quantification took place by HPLC.

### EMBODIMENT(S) OF THE INVENTION

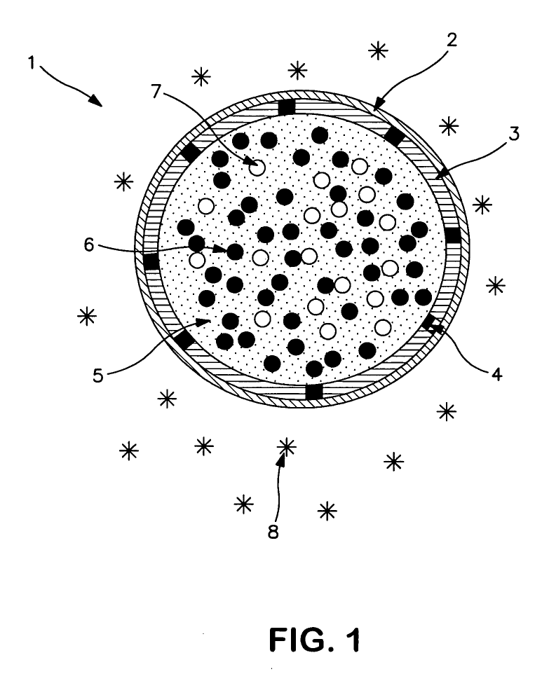

The solid pharmaceutical formulation I according to the invention (FIG. 1) comprises at least one layer 3 of one or more water-insoluble polymers, at least one layer 2 of one or more pH-dependently water-soluble polymers.

The formulation core 5 according to the invention is loaded with a strong pH-dependent water-soluble active ingredient 6 and at least one osmagent 7.

In a preferred embodiment of the invention, the layer 3 of one or more water-insoluble polymers comprises pore-forming substances 4 which are dissolved out after contact with the aqueous medium 8 and thus form a microporous membrane.

The one or more pore-forming substances 4 may be water-soluble polymers or other water-soluble additions such as salts or sugars.

The one or more pore-forming substances 4 may be selected from the group comprising for example polyvinylpyrrolidone (PVP), crospovidone (crosslinked N-vinyl-2-pyrrolidone, Cl-PVP), hydroxypropylmethylcellulose (HPMC), polyethylene glycol (PEG), hydroxypropylcellulose (HPC) and mixtures thereof.

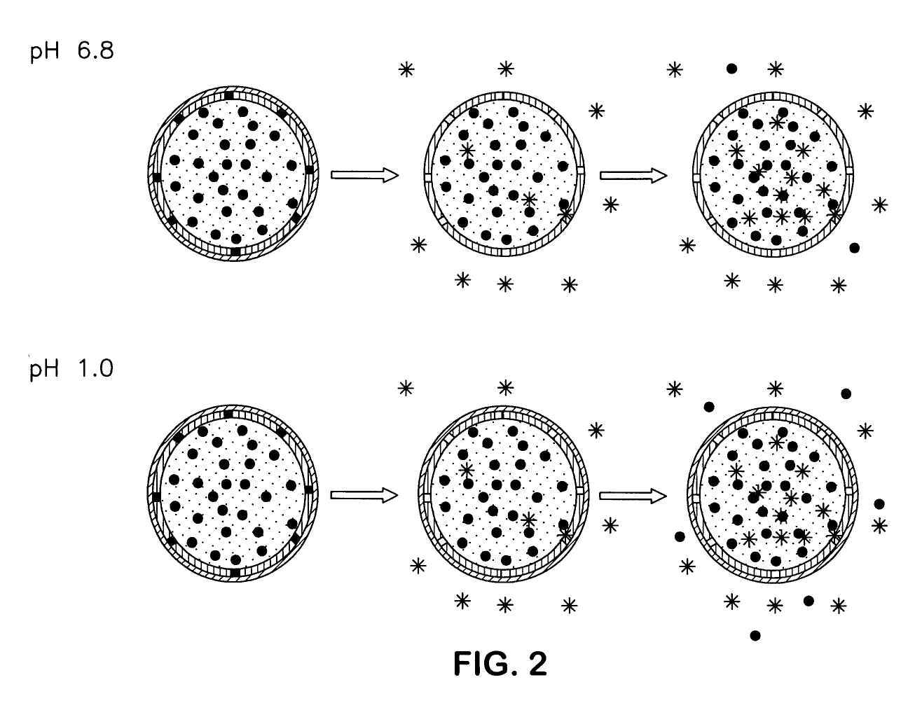

Formulations of an active ingredient-containing core without osmagent and of two layers of polymer (FIG. 2), where the inner layer consisted of a water-insoluble and the outer layer of a pH-dependently water-soluble polymer, still showed a strong pH-dependent release of the active substance.

The very greatly reduced active ingredient release in the medium with the actually highest active ingredient solubility was particularly noteworthy.

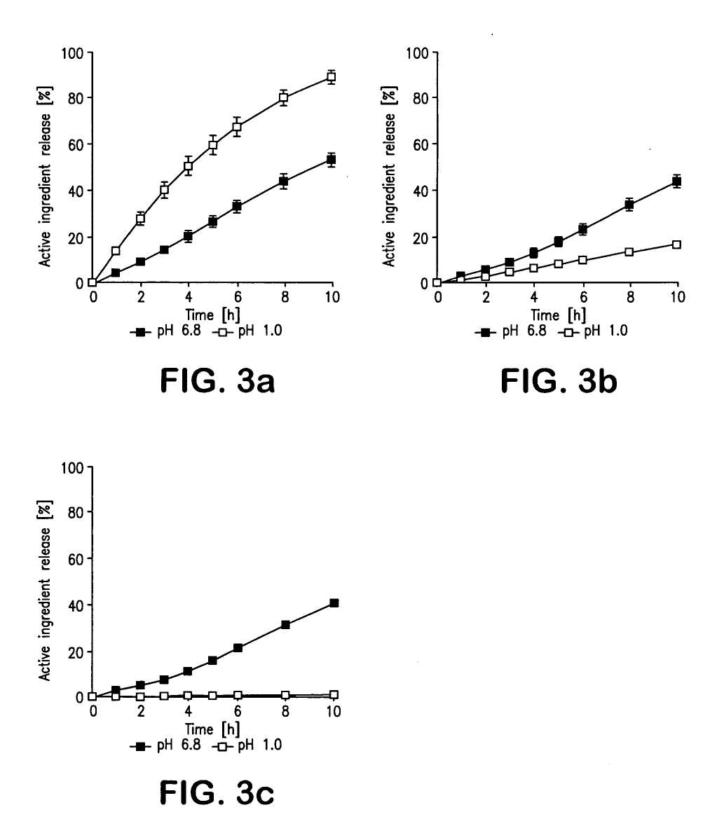

For example, a piperazineurea-containing core without osmagent according to FIG. 2 with higher solubility at acidic pH values (pH<4) was unable to achieve an efficient active ingredient release (FIG. 3a-c). The active ingredient release of piperazineurea in medium of pH 1 was less than expected.

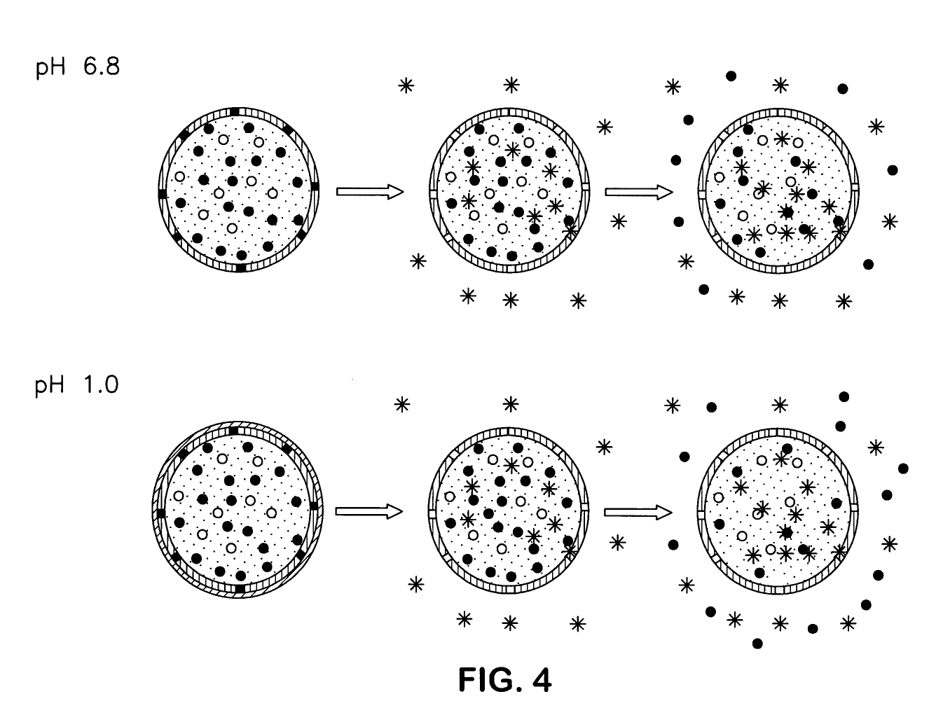

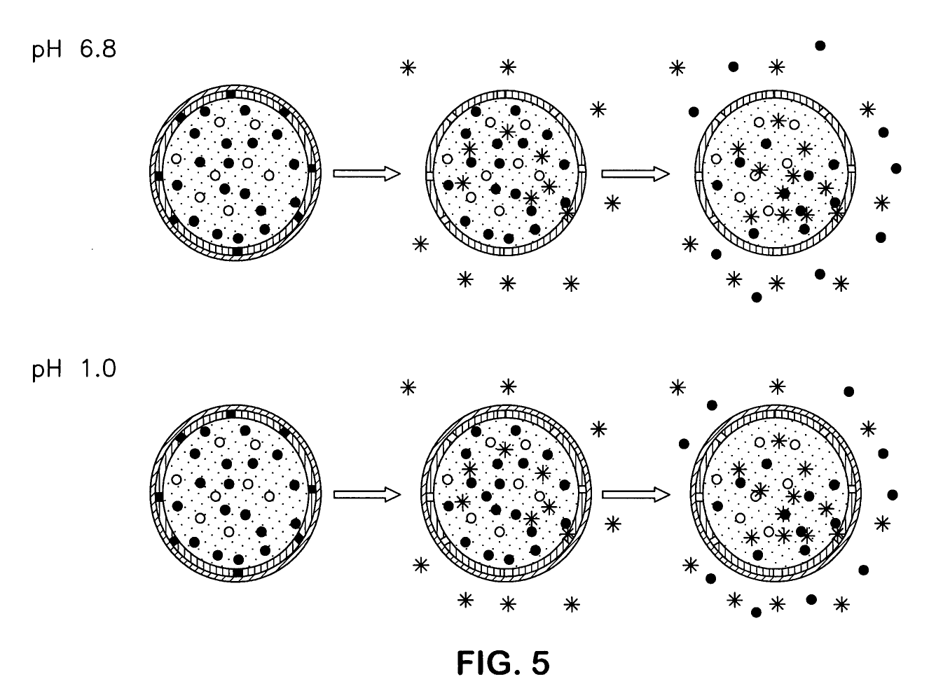

A pH-independently active ingredient release is not achieved even with a formulation without pH-dependently water-soluble polymer film (FIG. 4).

Efficient sustained pH-independent active ingredient releases of 0 or 1st order can easily be achieved by the solid pharmaceutical formulation according to the invention.

An example of the production of the solid pharmaceutical formulation for a sustained pH-independent active ingredient release according to the present invention is described below.

A dry powder mixture was prepared by introducing the sieved ingredients into a Müller drum with subsequent mixing in a Turbula mixer.

The dry powder mixture was subsequently moistened in a high-speed mixer, the amount of binder solution necessary for extrusion and spheronization having been determined by preliminary tests. The resulting moist granules were then extruded in an extruder and rounded in a spheronizer.

The produced pellets in a preferred embodiment of the invention were then dried in a fluidized bed (GPCG1 from Glatt).

After sieving, the pellet fraction from 0.8 mm to 1.25 mm diameter was used for further production.

The polymer dispersions were applied in a fluidized bed granulator with Wurster insert, with application of the first layer being followed by a brief drying pause and then application of the second layer.

The formulation layer of one or more water-insoluble polymers (subcoating formulation) is for example from 1% to 40% w/w, preferably from 1% to 10% w/w, preferably from 2% to 5% w/w based on the total mass of formulation. The water-insoluble polymers may be selected from the group comprising polyvinyl acetate; alkylcelluloses, acrylate-methacrylate copolymers, vinyl acetate-methacrylate copolymers and -acrylate copolymers; ethylcellulose, ethyl acrylate-methyl methacrylate copolymer and ethyl acrylate-methyl acrylate-trimethylammoniummethyl methacrylate chloride terpolymer and mixtures thereof.

In a preferred embodiment of the invention, pore-forming substances were used in the formulation layer of one or more water-insoluble polymers (subcoating formulation). The one or more pore-forming substances may be water-soluble polymers or other water-soluble additions such as salts or sugars. In a preferred embodiment of the invention, the one or more pore-forming substances may be selected from the group comprising for example polyvinylpyrrolidone (PVP), crospovidone (crosslinked N-vinyl-2-pyrrolidone, Cl-PVP), hydroxypropylmethylcellulose (HPMC), polyethylene glycol (PEG), hydroxypropylcellulose (HPC) and mixtures thereof.

The formulation layer of one or more pH-dependent water-soluble polymers (topcoating formulation) is for example from 1% to 40% w/w, preferably from 1% to 10% w/w, preferably from 2% to 5% w/w, based on the total mass of formulation.

The acid-insoluble polymers may be selected from the group comprising acrylate-methacrylic acid copolymers, carboxyalkylcelluloses, cellulose acetate phthalates, cellulose acetate succinates, cellulose acetate trimelliates, hydroxyalkylcellulose phthalates, hydroxyalkylcellulose acetate succinates, vinyl acetate phthalates, vinyl acetate succinate; ethylacrylate-methacrylic acid copolymer, methyl methacrylate-methacrylic acid copolymer, methyl methacrylate-methyl acrylate-methacrylic acid copolymer, carboxymethylcellulose, cellulose acetate phthalate; hydroxypropylmethylcellulose phthalate, hydroxypropylmethylcellulose acetate phthalate, hydroxypropylmethylcellulose acetate succinate, polyvinyl acetate phthalate, shellac and mixtures thereof.

Alkali-insoluble polymers which can be used are acrylate-methacrylate copolymers, basic natural polysaccharides, dimethylaminoethyl methacrylate-methyl methacrylate-butyl methacrylate terpolymer, chitosan and mixtures thereof.

Osmotically active substances (osmagents) which can be used for targeted pH-independent active ingredient release are water-soluble ionic or nonionic substances and hydrophilic polymers, alone or as mixture.

The water-soluble ionic substance may be selected from the group comprising magnesium chloride, magnesium sulphate, lithium chloride, sodium, chloride, potassium chloride, lithium sulphate, sodium sulphate, potassium sulphate, lithium phosphate, sodium phosphate, potassium phosphate, sodium carbonate, ammonium chloride, ammonium sulphate, ammonium phosphate alone or as mixture.

The content of water-soluble ionic osmotic substance in the core may be from 2% to 50% w/w based on the total mass of cores and in particular from 2% to 20% w/w based on the total mass of cores.

A water-soluble nonionic substance may be selected from the group comprising for example sucrose, mannitol, lactose, dextrose, sorbitol, alone or as mixture.

The content of water-soluble nonionic osmotic substance in the core may be from 2% to 50% w/w based on the total mass of cores and in particular from 10% to 40% w/w based on the total mass of cores.

The hydrophilic polymers may be selected from the group comprising hydroxypropylmethylcellulose (HPMC), hydroxypropylcellulose (HPC), xanthan gum, alginate, sodium carboxylmethylcellulose, polyvinylpyrrolidone (PVP), Cl-polyvinylpyrrolidone (Cl-PVP), polyethylene oxide, carbopols, polyacrylamides, gum arabic and mixtures thereof.

Water-soluble ionic substances preferably used according to the present invention are those which achieve a high osmotic effect with relatively small amounts.

It is possible to use cellulose or cellulose derivatives as additional formulating agent for influencing the mechanical strength of the pharmaceutical form. Microcrystalline cellulose is particularly advantageous.

### EXAMPLES

### Example 1

Production of coated matrix pellets with pH-dependent water-soluble polymer layer without osmagent in the core (FIG. 3a-c; state of the art)

Core Formulation (% w/w):

| | |
|---|---|
| Active ingredient (piperazineurea) | 60% |
| Microcrystalline cellulose | 40% |

Formulation Layer of One or More Water-insoluble Polymers (Subcoating Formulation) (% w/w):

| | |
|---|---|
| Polyvinyl acetate | 70% |
| Polyvinylpyrrolidone | 30% |
| Coating level of the | 5% w/w based on total mass of pellets. |
| subcoating formulation: | |

Formulation Layer of One or More pH-dependent Water-soluble Polymers (Topcoating Formulation) (% w/w):
Methacrylic acid-ethyl acrylate copolymer 100%
Coating level of the topcoating formulation (% w/w):
   0% (FIG. 3a); 2.5% (FIG. 3b); 5% (FIG. 3c) w/w based on total mass of pellets.

Microcrystalline cellulose and active ingredient are sieved and mixed in a Turbula mixer for 20 minutes.

The dry powder mixture is mixed with the required amount of binder solution (water) in a high-speed mixer. The resulting moist granules are subsequently extruded through a 1 mm screen in an extruder.

The produced extrudate is rounded in portions in a spheronizer at 400 rpm. The pellets are subsequently dried in a GPCGI fluidized bed granulator at 60° C.

After sieving, the pellet fraction from 0.8 mm to 1.25 mm diameter was used for further production.

The matrix pellet cores are equilibrated at 50° C. in a GPCGI fluidized bed granulator with Wurster insert for 10 minutes. Then a 15% (w/w) polyvinyl acetate dispersion which comprises the water-soluble pore former polyvinylpyrrolidone is applied at an inlet air temperature of 50° C.

After intermediate drying for 10 min, the pH-dependently soluble methacrylic acid-ethyl acrylate copolymer (15% w/w) is sprayed on at an inlet air temperature of 50° C.

After the polymer has been applied, the coated matrix pellets are equilibrated at 40° C. for 24 h.

### Example 2

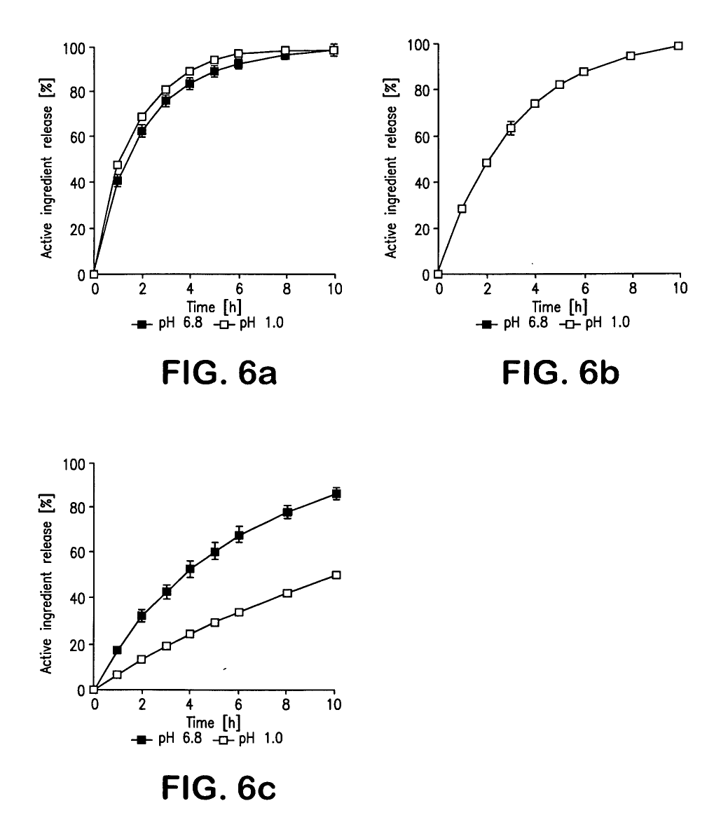

Production of coated matrix pellets with pH-dependent water-soluble polymer layer with osmagent (KCl) in the core (FIGS. 6a-c)

Core Formulation (% w/w):

| | |
|---|---|
| Active ingredient (piperazineurea) | 60% |
| Osmotically active substance (KCl) | 15% |
| Microcrystalline cellulose | 25% |

Formulation Layer of One or More Water-insoluble Polymers (Subcoating Formulation) (% w/w):

| | |
|---|---|
| Polyvinyl acetate | 70% |
| Polyvinylpyrrolidone | 30% |
| Coating level of the | 5% w/w based on total mass of pellets. |
| subcoating formulation: | |

Formulation Layer of One or More pH-dependent Water-soluble Polymers (Topcoating Formulation) (% w/w):
Methacrylic acid-ethyl acrylate copolymer 100%
Coating level of the topcoating formulation (% w/w):
   0% (FIG. 6a); 2.5% (FIG. 6b); 5% (FIG. 6c) based on total mass of pellets.

Microcrystalline cellulose and active ingredient are sieved and mixed in a Turbula mixer for 10 minutes. Sieved potassium chloride is added and mixed in the Turbula mixer for a further 10 minutes.

The dry powder mixture is mixed with the required amount of binder solution (water) in a high-speed mixer. The resulting moist granules are subsequently extruded through a 1 mm screen in an extruder.

The produced extrudate is rounded in portions in a spheronizer at 400 rpm. The pellets are subsequently dried in a GPCG1 fluidized bed granulator at 60° C.

After sieving, the pellet fraction from 0.8 mm to 1.25 mm diameter was used for further production.

The matrix pellet cores are equilibrated at 50° C. in a GPCGI fluidized bed granulator with Wurster insert for 10 minutes. Then a 15% (w/w) polyvinyl acetate dispersion which comprises the water-soluble pore former polyvinylpyrrolidone is applied at an inlet air temperature of 50° C.

After intermediate drying for 10 min, the pH-dependently soluble methacrylic acid-ethyl acrylate copolymer (15% w/w) is sprayed on at an inlet air temperature of 50° C.

After the polymer has been applied, the coated matrix pellets are equilibrated at 40° C. for 24h.

### Example 3

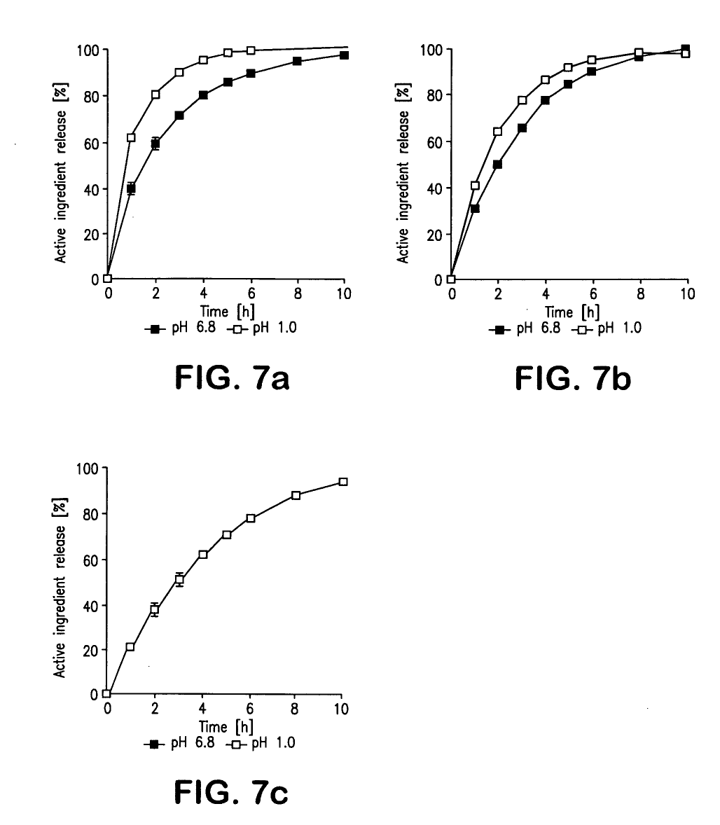

Production of coated matrix pellets with pH-dependent water-soluble polymer layer with osmagent (NaCl) in the core (FIGS. 7a-c)

Core Formulation (% w/w):

| | |
|---|---|
| Active ingredient (piperazineurea) | 60% |
| Osmotically active substance (NaCl) | 15% |
| Microcrystalline cellulose | 25% |

Formulation Layer of One or More Water-insoluble Polymers (Subcoating Formulation) (%

| | |
|---|---|
| Polyvinyl acetate | 70% |
| Polyvinylpyrrolidone | 30% |
| Coating level of the | 5% w/w based on total mass of |
| subcoating formulation: | pellets. |

Formulation Layer of One or More pH-dependent Water-soluble Polymers (Topcoating Formulation):
Methacrylic acid-ethyl acrylate copolymer 100%
Coating level of the topcoating formulation (% w/w):
   0% (FIG. 7a); 3% (FIG. 7b); 4% (FIG. 7c) based on total mass of pellets.

Microcrystalline cellulose and active ingredient are sieved and mixed in a Turbula mixer for 10 minutes. Sieved sodium chloride is added and mixed in the Turbula mixer for a further 10 minutes.

The dry powder mixture is mixed with the required amount of binder solution (water) in a high-speed mixer. The resulting moist granules are subsequently extruded through a 1 mm screen in an extruder.

The produced extrudate is rounded in portions in a spheronizer at 400 rpm. The pellets are subsequently dried in a GPCG1 fluidized bed granulator at 60° C.

After sieving, the pellet fraction from 0.8 mm to 1.25 mm diameter was used for further production.

The matrix pellet cores are equilibrated at 50° C. in a GPCG1 fluidized bed granulator with Wurster insert for 10 minutes. Then a 15% (w/w) polyvinyl acetate dispersion which comprises the water-soluble pore former polyvinylpyrrolidone is applied at an inlet air temperature of 50° C.

After intermediate drying for 10 min, the pH-dependently soluble methacrylic acid-ethyl acrylate copolymer (15% w/w) is sprayed on at an inlet air temperature of 50° C.

After the polymer has been applied, the coated matrix pellets are equilibrated at 40° C. for 24 h.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/706,428, filed Aug. 9, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Solid pharmaceutical formulation for a sustained pH-independent active ingredient release comprising
at least one layer of one or more water-insoluble polymers
at least one layer of one or more pH-dependent water-soluble polymers, and
an active ingredient-containing core, where
the core comprises an active ingredient having strong pH-dependent water solubility and at least one osmagent.

2. Solid pharmaceutical formulation according to claim 1, where the layer of one or more pH-dependent water-soluble polymers is the outer layer of the solid pharmaceutical formulation, and the layer of one or more water-insoluble polymers is the inner one.

3. Solid pharmaceutical formulation according to claim 1, where the layer of one or more water-insoluble polymers is from 1% to 40% w/w based on the total mass of the formulation.

4. Solid pharmaceutical formulation according to claim 1, where the layer of one or more water-insoluble polymers is from 1% to 10% w/w based on the total mass of the formulation.

5. Solid pharmaceutical formulation according to claim 1, where the layer of one or more water-insoluble polymers is from 2% to 5% w/w based on the total mass of the formulation.

6. Solid pharmaceutical formulation according to claim 1, where one or more water-insoluble polymers from the group comprising polyvinyl acetate, alkylcelluloses, acrylate-methacrylate copolymers, vinyl acetate-methacrylate copolymers and acrylate copolymers, ethylcellulose, ethyl acrylate-methyl methacrylate copolymer and ethyl acrylate-methyl acrylate-trimethylammoniummethyl methacrylate chloride terpolymer and mixtures thereof are selected.

7. Solid pharmaceutical formulation according to claim 1, where the layer of one or more water-insoluble polymers comprises one or more pore-forming substances.

8. Solid pharmaceutical formulation according to claim 1, where the pore-forming substance is selected from the group comprising polyvinyl-pyrrolidone, crospovidone, hydroxypropylmethylcellulose, polyethylene glycol, hydroxypropylcellulose and mixtures thereof.

9. Solid pharmaceutical formulation according to claim 1, where the layer of one or more pH-dependent water-soluble polymers is from 1% to 40% w/w based on the total mass of the formulation.

10. Solid pharmaceutical formulation according to claim 1, where the layer of one or more pH-dependent water-soluble polymers is from 1% to 10% w/w based on the total mass of the formulation.

11. Solid pharmaceutical formulation according to claim 1, where the layer of one or more pH-dependent water-soluble polymers is from 2% to 5% w/w based on the total mass of the formulation.

12. Solid pharmaceutical formulation according to claim 1, where one or more acid-insoluble polymers are selected from the group comprising
acrylate-methacrylic acid copolymers, carboxyalkylcelluloses, cellulose acetate phthalates, cellulose acetate succinates, cellulose acetate trimelliates,
hydroxyalkylcellulose phthalates, hydroxyalkylcellulose acetate succinates, vinyl acetate phthalates, vinyl acetate succinate; ethylacrylate-methacrylic acid copolymer, methyl methacrylate-methacrylic acid copolymer, methyl methacrylate-methyl acrylate-methacrylic acid copolymer, carboxymethylcellulose, cellulose acetate phthalate;
hydroxypropylmethylcellulose phthalate, hydroxypropylmethylcellulose acetate phthalate, hydroxypropylmethylcellulose acetate succinate, polyvinyl acetate phthalate, shellac and mixtures thereof.

13. Solid pharmaceutical formulation according to claim 1, where one or more alkali-insoluble polymers are selected from the group comprising acrylate-methacrylate copolymers, basic natural polysaccharides, dimethylaminoethyl methacrylate-methyl methacrylate-butyl methacrylate terpolymer, chitosan and mixtures thereof.

14. Solid pharmaceutical formulation according to claim 1, where the core is loaded with up to 90% w/w, based on the mass of the core formulation, of an active ingredient having strong pH-dependent water solubility.

15. Solid pharmaceutical formulation according to claim 1, where the core is loaded with up to 60% w/w, based on the mass of the core formulation, of an active ingredient having strong pH-dependent water solubility.

16. Solid pharmaceutical formulation according to claim 1, where the core is loaded with piperazineurea.

17. Solid pharmaceutical formulation according to claim 1, where the osmagent in the core is selected from the group comprising water-soluble ionic substances, water-soluble nonionic substances, hydrophilic polymers and mixtures thereof.

18. Solid pharmaceutical formulation according to claim 1, where the osmagent in the core is selected from the group comprising magnesium chloride, magnesium sulphate, lithium chloride, sodium chloride, potassium chloride, lithium sulphate, sodium sulphate, potassium sulphate, lithium phosphate, sodium phosphate, potassium phosphate, ammonium chloride, ammonium sulphate, ammonium phosphate, sucrose, mannitol, lactose, dextrose, sorbitol, hydroxypropylmethylcellulose, hydroxypropylcellulose, xanthan gum, alginate, sodium carboxylmethylcellulose, polyvinylpyrrolidone, Cl-polyvinylpyrrolidone, polyethylene oxide, carbopols, polyacrylamides, gum arabic and mixtures thereof.

19. Solid pharmaceutical formulation according to claim 1, which is a matrix pellet.

20. Solid pharmaceutical formulation according to claim 1, where the core has a diameter of from 0.8 mm to 1.25 mm.
